(19) United States
(12) Patent Application Publication
(10) Pub. No.: US 2024/0033309 A1
(43) Pub. Date: Feb. 1, 2024

(54) COMBINATION THERAPY FOR INFLAMMATORY DISORDERS OF THE JOINTS

(71) Applicant: SERVATUS LTD, COOLUM BEACH, QUEENSLAND (AU)

(72) Inventor: Wayne FINLAYSON, Peregian Springs, Queensland (AU)

(21) Appl. No.: 18/256,600

(22) PCT Filed: Dec. 8, 2021
(86) PCT No.: PCT/AU2021/051463

§ 371 Date: Jun. 8, 2023

(30) Foreign Application Priority Data
Dec. 9, 2020 (AU) 2020904573

(51) Int. Cl. A61K 35/747; A61K 39/395; A61K 31/519; A61P 19/02

(52) U.S. Cl. CPC A61K 35/747; A61K 39/3955; A61K 31/519; A61P 19/02

(57) ABSTRACT

Methods for treating inflammatory disorders of the joints, optionally rheumatoid arthritis, or at least one symptom thereof, including administering to a subject in need thereof an effective amount of: an immunosuppressant; and one or more Lactobacillus species selected from Lactobacillus buchneri, Lactobacillus paracasei, Lactobacillus zeae, Lactobacillus rapi, Lactobacillus parafarraginis, and Lactobacillus diolivorans, and/or a culture supernatant or cell free filtrate derived from culture media in which the one or more Lactobacillus species has been cultured. In particular embodiments, the method includes the administration of a combination of an immunosuppressant, Lactobacillus buchneri, Lactobacillus paracasei and Lactobacillus zeae.

### FIELD OF THE ART

The present disclosure relates generally to methods for the treatment or prevention of inflammatory disorders of the joints, optionally rheumatoid arthritis.

### BACKGROUND

Inflammation is a normal response mechanism assisting in protecting the body from infection and injury. However abnormal or uncontrolled inflammatory responses can result in the development of acute or chronic inflammatory and autoimmune disorders or conditions. In particular, infections caused by viruses, fungi and pathogenic bacteria can trigger excessive and persistent inflammatory responses in a variety of tissues, such as of the gastrointestinal tract, joints, skin and the urinary tract, leading to deleterious acute inflammation and acute inflammatory conditions. These are also a significant risk factor in the development of chronic inflammatory and autoimmune conditions. Chronic inflammatory and autoimmune conditions can be debilitating and cause enormous discomfort and pain to sufferers. Moreover, such conditions are increasing in prevalence as populations around the world age.

Rheumatoid arthritis is a chronic autoimmune disease affecting approximately 1% of the world's population. It is characterized by inflammation and cellular proliferation in the synovial lining of joints that can ultimately result in cartilage and bone destruction, joint deformity and loss of mobility. Rheumatoid arthritis usually causes problems in several joints at the same time, often in a symmetric manner. Early rheumatoid arthritis tends to affect the smaller joints first, such as the joints in the wrists, hands, ankles and feet. As the disease progresses, joints of the shoulders, elbows, knees, hips, jaw and neck can also become involved. Rheumatoid arthritis is a heterogeneous disease with limited, broadly efficacious treatment options. Currently there is no cure, and treatment is essentially directed towards relieving pain, reducing inflammation, and stopping or slowing joint damage and bone destruction.

Steroids have been the primary therapeutic anti-inflammatory agent relied upon for many decades. More recently non-steroidal anti-inflammatory drugs (NSAIDs) have begun to be commonly employed to manage or treat inflammation. However, the continued use of such agents comes with significant disadvantages and side effects. For example, associated with continued NSAID use are significant side effects including stomach ulcers and bleeding. Additionally, it is well known that NSAIDs produce lesions in the gastrointestinal tract, depending on the length of the treatment and on the type of drug. This problem is of particular importance in cases where the therapy must be protracted for a long time, such as in the treatment of chronic inflammatory disorders where long term treatment is needed to manage the inflammatory state and associated pain.

More recently, advances have led to the development of novel approaches to the treatment of rheumatoid arthritis. Tofacitinib is a small molecule, and a potent selective inhibitor of Janus kinase (JAK) 1 and JAK3 and, to a lesser extent, JAK2. JAKs mediate signal transduction activity by the surface receptors for multiple cytokines, including several interleukins Tofacitinib (Xeljanz®) is typically administered orally, twice daily and is indicated for the treatment of moderate to severe active rheumatoid arthritis, in particular in patients who have responded inadequately to one or more conventional disease-modifying antirheumatic drugs (such as methotrexate, leflunomide, hydroxychloroquine, and sulfasalazine). An alternative approach to the treatment of rheumatoid arthritis has been to employ tumour necrosis factor (TNF) inhibitors such as the monoclonal antibody adalimumab. Adalimumab (Humira®) is a recombinant IgG1 antibody that binds specifically to and neutralizes TNFα. Administered by subcutaneous injection as a monotherapy or in conjunction with methotrexate, adalimumab is indicated for the treatment of moderate to severe active rheumatoid arthritis, in particular in patients who have responded inadequately to one or more conventional disease-modifying antirheumatic drugs.

Despite recent improvements, treatment for rheumatoid arthritis remains inadequate in many instances. There is a continuing need for the development of new and improved therapeutic options for the treatment of inflammatory conditions of the joints such as rheumatoid arthritis.

### SUMMARY OF THE DISCLOSURE

One aspect of the present disclosure provides a method for treating an inflammatory disorder of the joints or at least one symptom thereof, comprising administering to a subject in need thereof an effective amount of:
(i) an immunosuppressant; and
(ii) one or more Lactobacillus species selected from Lactobacillus buchneri, Lactobacillus paracasei, Lactobacillus zeae, Lactobacillus rapi, Lactobacillus parafarraginis, and Lactobacillus diolivorans, and/or a culture supernatant(s) or cell free filtrate(s) derived from culture media in which said one or more Lactobacillus species has been cultured.

Typically the inflammatory disorder of the joints is an inflammatory arthritis. The inflammatory arthritis may be rheumatoid arthritis, psoriatic arthritis or ankylosing spondylitis. In a particular embodiment, the inflammatory disorder of the joints is rheumatoid arthritis.

In an embodiment, the immunosuppressant is a TNF inhibitor. In an exemplary embodiment the TNF inhibitor is adalimumab.

In an embodiment, the immunosuppressant is a JAK inhibitor. In an exemplary embodiment the JAK inhibitor is tofacitinib.

The one or more Lactobacillus species may comprise a combination of at least three of said Lactobacillus species, optionally comprising L. buchneri, L. paracasei and L. zeae. Thus, in an embodiment, the method comprises administering to the subject a combination of L. buchneri, L. paracasei and L. zeae or culture supernatant(s) or cell free filtrate(s) therefrom.

In an embodiment, the method comprises administering to the subject an effective amount of a TNF inhibitor, optionally adalimumab, and a combination of L. buchneri, L. paracasei and L. zeae or culture supernatant(s) or cell free filtrate(s) therefrom.

In another embodiment, the method comprises administering to the subject an effective amount of a JAK inhibitor, optionally tofacitinib, and a combination of L. buchneri, L. paracasei and L. zeae or culture supernatant(s) or cell free filtrate(s) therefrom.

The immunosuppressant and the one or more Lactobacillus species, culture supernatant(s) or cell free filtrate(s) therefrom, may be formulated in the same composition for administration. Alternatively, the immunosuppressant and the one or more Lactobacillus species may be administered in separate compositions. Such separate administration may be sequential or simultaneous.

The immunosuppressant and the one or more Lactobacillus species, culture supernatant(s) or cell free filtrate(s) therefrom, may be administered by the same or different routes, for example, orally, sublingually, topically or parenteral.

Another aspect of the present disclosure provides the use of:
(i) an immunosuppressant; and
(ii) one or more Lactobacillus species selected from Lactobacillus buchneri, Lactobacillus paracasei. Lactobacillus zeae. Lactobacillus rapi. Lactobacillus parafarraginis, and Lactobacillus diolivorans, and/or a culture supernatant(s) or cell free filtrate(s) derived from culture media in which said one or more Lactobacillus species has been cultured,
   in the manufacture of a medicament for the treatment of an inflammatory disorder of the joints or at least one symptom thereof.

In a particular embodiment, the immunosuppressant is selected from a TNF inhibitor, optionally adalimumab, and a JAK inhibitor, optionally tofacitinib.

In a particular embodiment, the one or more Lactobacillus species comprise a combination of L. buchneri, L. paracasei and L. zeae. Thus, in an embodiment, the medicament comprises a combination of L. buchneri, L. paracasei and L. zeae or culture supernatant(s) or cell free filtrate(s) therefrom.

In an embodiment, the medicament comprises a TNF inhibitor, optionally adalimumab, and a combination of L. buchneri, L. paracasei and L. zeae or culture supernatant(s) or cell free filtrate(s) therefrom.

In an embodiment, the medicament comprises a JAK inhibitor, optionally tofacitinib, and a combination of L. buchneri, L. paracasei and L. zeae or culture supernatant(s) or cell free filtrate(s) therefrom.

In accordance with the above aspects and embodiments, and as described and exemplified herein, typically the combination of the immunosuppressant and the one or more Lactobacillus species is a synergistic combination.

In accordance with aspects and embodiments of the present disclosure, the method may comprise administering to the subject a microbial biotherapeutic composition comprising L. buchneri, L. paracasei and L. zeae. The microbial biotherapeutic composition may be administered in the form of, for example, a liquid or solid unit dosage form, a food or a beverage.

### BRIEF DESCRIPTION OF THE DRAWINGS

Exemplary embodiments of the present disclosure are described herein, by way of non-limiting example only, with reference to the following drawings.

FIG. 1. Clinical score (paw volume summed for 4 paws) in mice of a collagen antibody-induced arthritis (CAIA) mouse model following treatment as described in Example 1. Numbers 1 to 7 (indicated by dashed lines) represent treatment groups 1 to 7, respectively, of Example 1: 1—naïve (negative control); 2—CAIA control; 3—CAIA+SVT combination; 4—CAIA+tofacitinib; 5—CAIA+tofacitinib+SVT combination; 6—CAIA+adalimumab; 7—CAIA+adalimumab+SVT combination.

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein the term “effective amount” includes within its meaning a non-toxic but sufficient amount of composition to provide the desired therapeutic effect. The exact amount required will vary from subject to subject depending on factors such as the species being treated, the age and general condition of the subject, the severity of the condition being treated, the particular agent being administered and the mode of administration and so forth. For any given case, an appropriate “effective amount” may be determined by one of ordinary skill in the art using only routine experimentation.

The term “subject” as used herein refers to mammals and includes humans, primates, livestock animals (e.g. cattle, dairy cows, horses, sheep, pigs), laboratory test animals (e.g. mice, rabbits, rats, guinea pigs), companion animals (e.g. dogs, cats), performance animals (e.g. racehorses), and captive wild animals. In exemplary embodiments, the mammal is human.

As used herein the terms “treating”, “treatment” and the like refer to any and all applications which remedy, or otherwise hinder, retard, or reverse the progression of, an inflammatory disorder of the joints, or at least one symptom of such a disorder, including reducing the severity of the disease. Thus, treatment does not necessarily imply that a subject is treated until complete elimination of, or recovery from, the disease.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

In the context of this specification, the term “microbial biotherapeutic” is to be given its broadest construction and is understood to refer to a microbial cell population or preparation, or component of a microbial cell population or preparation, which when administered to a subject in an effective amount promotes a health benefit in the subject.

In the context of this specification, the term “prebiotic” is to be given its broadest construction and is understood to refer to any non-digestible substance that stimulates the growth and/or activity of commensal beneficial bacteria in the digestive system.

In the context of this specification, the terms “food”, “foods”, “beverage” or “beverages” include but are not limited to health foods and beverages, functional foods and beverages, and foods and beverages for specified health use. When such foods or beverages of the present invention are used for subjects other than humans, the terms can be used to include a feedstuff.

Provided herein are methods for treating an inflammatory disorder of the joints or at least one symptom thereof, comprising administering to a subject in need thereof an effective amount of:
(i) an immunosuppressant; and
(ii) one or more Lactobacillus species selected from Lactobacillus buchneri, Lactobacillus paracasei, Lactobacillus zeae, Lactobacillus rapi, Lactobacillus parafarraginis, and Lactobacillus diolivorans, and/or a culture supernatant or cell free filtrate derived from culture media in which said one or more Lactobacillus species has been cultured.

The inflammatory disorder to which methods of the present disclosure relate is typically an inflammatory arthritis. The inflammatory arthritis may be selected from, for example, rheumatoid arthritis, psoriatic arthritis and ankylosing spondylitis. In particular embodiments, the inflammatory disorder is rheumatoid arthritis.

Embodiments of the present disclosure may comprise reducing the expression of one or more pro-inflammatory cytokines in a subject suffering from an inflammatory disorder of the joints, wherein the reduction observed is relative to the level of expression of the pro-inflammatory cytokines observed in the subject in the absence of said treatment. Such reduction may comprise normalization of the level of expression of the pro-inflammatory cytokines. Exemplary pro-inflammatory cytokines include interleukins such as IL-6 and IL-1β, KC-GRO (keratinocyte chemoattractant/human growth-regulated oncogene) and TNFα.

The methods of the present disclosure may inhibit inflammation associated with the inflammatory disorder. The term “inhibit” and variations thereof such as “inhibition”, “inhibits”, “reduces”, “reducing” and the like, are used interchangeably herein to denote an improvement (i.e., reduction) in the severity of inflammation associated with the inflammatory disorder.

Methods of the present disclosure employ the administration of an immunosuppressant in combination with one or more Lactobacillus species selected from Lactobacillus buchneri, Lactobacillus paracasei, Lactobacillus zeae, Lactobacillus rapi, Lactobacillus parafarraginis, and Lactobacillus diolivorans, and/or a culture supernatant or cell free filtrate derived from culture media in which said one or more Lactobacillus species has been cultured. As exemplified herein, typically the combination of the immunosuppressant and the one or more Lactobacillus species is a synergistic combination.

In particular embodiments the immunosuppressant may be, for example, a TNF inhibitor, a JAK inhibitor or a calcineurin inhibitor. Suitable TNF inhibitors include but are not limited to monoclonal antibodies such as adalimumab, infliximab, natalizumab, and biosimilars thereof, but does not include etanercept. The JAK inhibitor may be a selective or non-selective inhibitor and may be, for example, a JAK1 inhibitor, a JAK2 inhibitor, a JAK1/JAK2 inhibitor or a JAK3 inhibitor. Exemplary JAK inhibitors include but are not limited to tofacitinib, baracitinib, upadacitinib, ruxolitinib, oclacitinib, peficitinib and fedracitinib. Suitable calcineurin inhibitors include but are not limited to cyclosporin A, tacrolimus and analogues thereof.

In particular embodiments, the immunosuppressant is one that is known to have at least partial efficacy, when used as a sole therapeutic agent, in the treatment of inflammatory disorders of the joints such as rheumatoid arthritis.

In an exemplary embodiment the immunosuppressant is a TNF inhibitor, optionally adalimumab, or a JAK inhibitor, optionally tofacitinib.

Methods of the present disclosure employ the administration of one or more Lactobacillus species selected from Lactobacillus buchneri, Lactobacillus paracasei, Lactobacillus zeae, Lactobacillus rapi, Lactobacillus parafarraginis, and Lactobacillus diolivorans and/or a culture supernatant(s) or cell free filtrate(s) derived from culture media in which said one or more Lactobacillus species has been cultured. In view of some taxonomic discrepancies and uncertainties, Lactobacillus zeae may also be referred to elsewhere as Lactobacillus casei. However this is not settled, and L. zeae can be regarded as distinct (see http://lactotax.embl.de/wuyts/lactotax/). For the purposes of the present disclosure the L. zeae nomenclature is retained.

In some embodiments the methods of the present disclosure contemplate the administration of one or more Lactobacillus species selected from Lactobacillus buchneri, Lactobacillus paracasei, Lactobacillus zeae, Lactobacillus rapi, Lactobacillus parafarraginis, and Lactobacillus diolivorans, in the same or different compositions. In some embodiments the methods of the present disclosure contemplate the administration of Lactobacillus buchneri, Lactobacillus paracasei and Lactobacillus zeae, in the same or different compositions.

In the following discussion, in the context of administration of the Lactobacillus species or culture supernatants or cell free filtrates derived from culture media in which Lactobacillus has been cultured, and in the context of compositions comprising the same, the term “Lactobacillus” may be used to refer not only to the specific Lactobacillus species defined herein per se, but also more broadly to refer to culture supernatants or cell free filtrates derived from culture media in which the specific Lactobacillus species defined herein have been cultured.

Methods of the present disclosure may comprise the administration of any two, three, four, five or all six of the Lactobacillus species Lactobacillus buchneri, Lactobacillus paracasei, Lactobacillus zeae, Lactobacillus rapi, Lactobacillus parafarraginis, and Lactobacillus diolivorans, or culture supernatants or cell free filtrates derived from culture media in which two, three, four, five or all six of said Lactobacillus have been cultured. In such embodiments the bacteria may be cultured separately or together. Accordingly, the administration may comprise administration of a composition comprising a combination of two, three, four, five or all six of the Lactobacillus species described herein. Similarly, where culture supernatants or cell free filtrates derived from culture media in which two, three, four, five or all six of said Lactobacillus have been cultured are administered, the culture supernatants or cell free filtrates may be derived from the culturing of Lactobacillus species individually, said supernatants or cell free filtrates being combined prior to administration, or may be derived from a combined culture of two, three, four, five or all six of the Lactobacillus species described herein.

In an exemplary embodiment, the methods of the present disclosure comprise the administration of a combination of Lactobacillus buchneri, Lactobacillus paracasei, Lactobacillus zeae, or a culture supernatant(s) or cell free filtrate(s) thereof. In a particular exemplary embodiment, the methods of the present disclosure comprise the administration of a combination of Lactobacillus buchneri, Lactobacillus paracasei, Lactobacillus zeae.

Also provided herein are methods for treating an inflammatory disorder of the joints or at least one symptom thereof, comprising administering to a subject in need thereof an effective amount of a combination of Lactobacillus buchneri, Lactobacillus paracasei and Lactobacillus zeae or a culture supernatant(s) or cell free filtrate(s) derived from culture media in which said Lactobacillus species have been cultured. Optionally, a combination of Lactobacillus buchneri, Lactobacillus paracasei and Lactobacillus zeae is administered.

The Lactobacillus buchneri may be Lactobacillus buchneri Lb23 available under Accession Number V11/022946, previously described in WO2013/063658. The L. buchneri may be L. buchneri SVT 06B1 (which may be elsewhere referred to by the alternate designation SVT-23) deposited pursuant to the Budapest Treaty with the Belgian Co-Ordinated Collections of Micro-organisms (BCCM) on 27 Feb. 2019 under Accession Number LMG P-31293.

The Lactobacillus paracasei may be Lactobacillus paracasei Lp9 available under Accession Number V12/022849, previously described in WO2014/172758 (designated as strain ‘T9’ therein). The Lactobacillus paracasei may be Lactobacillus paracasei SVT 04P1 (which may be elsewhere referred to by the alternate designation SVT-09) deposited pursuant to the Budapest Treaty with the Belgian Co-Ordinated Collections of Micro-organisms (BCCM) on 27 Feb. 2019 under Accession Number LMG P-31290.

The Lactobacillus zeae may be Lactobacillus zeae Lz26 available under Accession Number V11/022948, previously described in WO2013/063658. The Lactobacillus zeae may be Lactobacillus zeae SVT 08Z1 (which may be elsewhere referred to by the alternate designation SVT-26) deposited pursuant to the Budapest Treaty with the Belgian Co-Ordinated Collections of Micro-organisms (BCCM) on 27 Feb. 2019 under Accession Number LMG P-31295.

The Lactobacillus rapi may be Lactobacillus rapi Lr24 available under Accession Number V11/022947, previously described in WO2013/063658. The Lactobacillus rapi may be Lactobacillus rapi SVT 07R1 (which may be elsewhere referred to by the alternate designation SVT-24) deposited pursuant to the Budapest Treaty with the Belgian Co-Ordinated Collections of Micro-organisms (BCCM) on 27 Feb. 2019 under Accession Number LMG P-31294.

The Lactobacillus parafarraginis may be Lactobacillus parafarraginis Lp18 available under Accession Number V11/022945, previously described in WO2013/063658. The Lactobacillus parafarraginis may be Lactobacillus parafarraginis SVT 05P2 (which may be elsewhere referred to by the alternate designation SVT-18) deposited pursuant to the Budapest Treaty with the Belgian Co-Ordinated Collections of Micro-organisms (BCCM) on 27 Feb. 2019 under Accession Number LMG P-31292.

The Lactobacillus diolivorans may be Lactobacillus diolivorans Ld3 available under Accession Number V12/022847, previously described in WO2014/172758 (designated as strain ‘N3’ therein). The Lactobacillus diolivorans may be Lactobacillus diolivorans SVT 01D1 (which may be elsewhere referred to by the alternate designation SVT-03) deposited pursuant to the Budapest Treaty with the Belgian Co-Ordinated Collections of Micro-organisms (BCCM) on 27 Feb. 2019 under Accession Number LMG P-31287.

Where Lactobacillus organisms per se are administered, the concentrations of individual Lactobacillus species to be administered in accordance with methods of the present disclosure will depend on a variety of factors including the identity and number of individual species employed, the exact nature and severity of the inflammatory disorder to be treated, the form in which a composition is applied and the means by which it is applied. For any given case, appropriate concentrations may be determined by one of ordinary skill in the art using only routine experimentation. By way of example only, the concentration of the Lactobacillus species, or each species present in the case of a combination, may be from about 1×10² cfu/ml to about 1×10¹¹ cfu/ml, and may be about 1×10³ cfu/ml, about 2.5×10³ cfu/ml, about 5×10³ cfu/ml, 1×10⁴ cfu/ml, about 2.5×10⁴ cfu/ml, about 5×10⁴ cfu/ml, 1×10⁵ cfu/ml, about 2.5×10⁵ cfu/ml, about 5×10⁵ cfu/ml, 1×10⁶ cfu/ml, about 2.5×10⁶ cfu/ml, about 5×10⁶ cfu/ml, 1×10⁷ cfu/ml, about 2.5×10⁷ cfu/ml, about 5×10⁷ cfu/ml, 1×10⁸ cfu/ml, about 2.5×10⁸ cfu/ml, about 5×10⁸ cfu/ml, 1×10⁹ cfu/ml, about 2.5×10⁹ cfu/ml, or about 5×10⁹ cfu/ml, about 1×10¹⁰ cfu/ml, about 1.5×10¹⁰ cfu/ml, about 2.5×10¹⁰ cfu/ml, about 5×10¹⁰ cfu/ml or about 1×10¹¹ cfu/ml.

Also contemplated by the present disclosure is the use of variants of the Lactobacillus species described herein. As used herein, the term “variant” refers to both naturally occurring and specifically developed variants or mutants of the species disclosed and exemplified herein. Variants may or may not have the same identifying biological characteristics of the specific species exemplified herein, provided they share similar advantageous properties in terms of treating or preventing inflammatory conditions. Illustrative examples of suitable methods for preparing variants exemplified herein include, but are not limited to, gene integration techniques such as those mediated by insertional elements or transposons or by homologous recombination, other recombinant DNA techniques for modifying, inserting, deleting, activating or silencing genes, intraspecific protoplast fusion, mutagenesis by irradiation with ultraviolet light or X-rays, or by treatment with a chemical mutagen such as nitrosoguanidine, methylmethane sulfonate, nitrogen mustard and the like, and bacteriophage-mediated transduction. Suitable and applicable methods are well known in the art and are described, for example, in J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); and J. Sambrook, D. Russell, Molecular Cloning: A Laboratory Manual, 3rd ed., Cold Spring Harbor Laboratory Press, Cold Spring Harbor, N.Y. (2001), inter alia.

Also encompassed by the term “variant” as used herein are microbial strains phylogenetically closely related to the Lactobacillus species described herein and strains possessing substantial sequence identity with the species described herein at one or more phylogenetically informative markers such as rRNA genes, elongation and initiation factor genes, RNA polymerase subunit genes, DNA gyrase genes, heat shock protein genes and recA genes. For example, the 16S rRNA genes of a “variant” strain as contemplated herein may share about 85%, 86%, 87%, 88%, 89%, 90%, 91%, 92%, 93%, 94%, 95%, 96%, 97%, 98% or 99% sequence identity with a strain disclosed herein.

The Lactobacillus species described herein, and combinations thereof, or culture supernatants or cell free filtrates derived from culture media are typically administered in accordance with the present disclosure in the form of a composition. In embodiments in which combinations of species, or culture supernatants or cell free filtrates derived from culturing multiple species, those skilled in the art will appreciate that each of the species, supernatants or filtrates to be administered need not be contained in the same composition. Where administration is separate, administration may be sequential or simultaneous.

Similarly, the immunosuppressant may be administered in the same composition as one or more of the Lactobacillus species or culture supernatant(s) or cell free filtrate(s), or as the one or more Lactobacillus species or culture supernatant(s) or cell free filtrate(s), or may be administered in a different composition. Where the immunosuppressant is present in a different composition, the compositions may be administered sequentially or simultaneously.

Compositions for use in accordance with the present disclosure may be prepared by admixing the relevant components and formulating the resulting mixture into a dosage form that is suitable for administration to a subject. Accordingly, the compositions may comprise pharmaceutically acceptable carriers, diluents, excipients and/or adjuvants. The carriers, diluents, excipients and adjuvants must be “acceptable” in terms of being compatible with other components of the composition, and not deleterious to the subject who is to receive the composition. Methods for preparing suitable compositions for administration, and carriers, diluents, excipients and adjuvants suitable for use in compositions formulated for topical, oral or sublingual administration are well known to those skilled in the art. In exemplary embodiments, the composition may comprise one or more microbial biotherapeutic strains concentrated (e.g. by centrifugation and/or filtration) following cell culture to remove excess media. As such, the composition may comprise one or more microbial biotherapeutic strains in residual food grade media. Alternatively, the composition may be formulated with a carrier comprising sterile isotonic saline or 3% sucrose.

Compositions may be administered via any convenient or suitable route, variety of routes including, but not limited to, oral, sublingual, buccal, rectal, topical, intranasal, intraocular, transmucosal, intestinal, enteral, intramuscular, subcutaneous, intramedullary, intrathecal, intraventricular, intracerebral, intravesical, intravenous or intraperitoneal. The appropriate route may depend, for example, on the nature and severity of the inflammatory disorder to be treated. Where the immunosuppressant is administered in a different composition to the one or more Lactobacillus species or culture supernatant(s) or cell free filtrate(s), the route of administration of the compositions may be the same or different.

By way of example only: compositions comprising the one or more Lactobacillus species or a culture supernatant or cell free filtrate derived from culture media in which said one or more Lactobacillus species has been cultured may be administered orally; and compositions comprising the immunosuppressant may be administered orally or by injection. For example, compositions comprising tofacitinib may be administered orally, and compositions comprising adalimumab may be administered by subcutaneous injection.

Accordingly, methods of the present disclosure contemplate the administration of components of the combinations described in the same or different compositions, and via the same or different routes. Exemplary embodiments of methods of the disclosure comprise the oral administration of one or more Lactobacillus strains and an immunosuppressant such as tofacitinib, wherein the Lactobacillus strains and the CsA or tofacitinib are in the same or different compositions. Exemplary embodiments of methods of the disclosure comprise the oral administration of one or more Lactobacillus strains and the administration of an immunosuppressant such as adalimumab by injection, optionally subcutaneous injection.

Compositions may be administered in accordance with the present disclosure in any suitable form, typically in solid or liquid form. For example, the compositions may be formulated using methods and techniques well known to those skilled in the art, into tablets, troches, capsules, caplets, elixirs, suspensions, syrups, wafers, granules, powders, gels, pastes, solutions, creams, sprays, suspensions, soluble sachets, lozenges, effervescent tablets, chewable tablets, multi-layer tablets, and the like. For oral administration, the Lactobacillus or compositions may be conveniently incorporated in a variety of beverages, food products, nutraceutical products, nutritional supplements, food additives, pharmaceuticals, over-the-counter formulations and animal feed supplements. For topical application, suitable vehicles include, but are not limited to, lotions, liniments, gels, creams, ointments, foams, sprays, oils, powders and the like. Compositions may also be impregnated into transdermal patches, plasters, and wound dressings such as bandages or hydrocolloid dressings, typically in liquid or semi-liquid form.

As will be appreciated by those skilled in the art, the choice of pharmaceutically acceptable carrier or diluent will be dependent on the route of administration and on the nature and severity of the condition and the subject to be treated. The particular carrier or delivery system and route of administration may be readily determined by a person skilled in the art. A person skilled in the art will readily be able to determine appropriate formulations useful in the methods of the disclosure using conventional approaches.

For example, compositions of the present disclosure may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

The Lactobacillus can be formulated readily using pharmaceutically acceptable carriers well known in the art into dosages suitable for oral administration. These carriers may be selected from sugars, starches, cellulose and its derivatives, malt, gelatine, talc, calcium sulfate, vegetable oils, synthetic oils, polyols, alginic acid, phosphate buffered solutions, emulsifiers, isotonic saline and pyrogen-free water.

Some examples of suitable carriers, diluents, excipients and adjuvants for oral use in accordance with the present disclosure include liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration. Adjuvants typically include emollients, emulsifiers, thickening agents, preservatives, bactericides and buffering agents. For administration as an injectable solution or suspension, non-toxic parenterally acceptable diluents or carriers can include, Ringer's solution, isotonic saline, phosphate buffered saline, ethanol and 1,2 propylene glycol.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof. Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like. Emulsions for oral administration may further comprise one or more emulsifying agents. Suitable emulsifying agents include dispersing agents as exemplified above or natural gums such as guar gum, gum acacia or gum tragacanth.

Methods for preparing suitable parenterally administrable compositions will be well known to those skilled in the art, and are described in more detail in, for example, Remington's Pharmaceutical Science, 15th ed., Mack Publishing Company, Easton, Pa., hereby incorporated by reference herein.

For compositions formulated for topical administration, examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenylpolysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly.

In further embodiments, the composition may further comprise suspending agents and/or humectants, such as povidone or propylene glycol, and neutralising agents for adjusting the viscosity of the composition, such as sodium hydroxide, triethanolamine (TEA) or ethylenediamine tetraacetic acid (EDTA).

Compositions of the present disclosure may be administered, for example one or more times a week, optionally for example once a week, once every second day, once a day, twice a day or three times a day, depending on the condition to be treated or prevented, the severity of the condition and the desired outcome. The duration of administration by a subject will also vary depending on the condition to be treated or prevented, the severity of the condition and the desired outcome. The amount of composition to be administered by a subject will vary depending on a range of factors including the identity of the microorganisms administered, the nature and severity of the condition to be treated or prevented, the age and general wellbeing of the subject, and the desired outcome. Suitable dosage regimes can readily be determined by the skilled addressee.

In exemplary embodiments, about 1 ml to about 25 ml liquid formulation of a Lactobacillus species at a final concentration of between about 10⁵ and 10¹¹ cfu/ml may be administered to a subject on a once-a-day, twice-a-day or more frequent basis. The volume of the liquid formulation may be, for example, about 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, or 25 ml.

The combination of immunosuppressant and one or more Lactobacillus species or culture supernatant(s) or cell free filtrate(s) may be administered in conjunction with one or more other therapeutic agents for example, but not limited to, antibiotics, antimicrobial agents, antiseptics, anaesthetics, anti-inflammatory agents, immunosuppressive agents and other therapeutic agents indicated for the treatment of inflammatory conditions such as steroids, and NSAIDs. Administration of such additional agents may be at the same time or at different times, i.e. simultaneous or sequential, and may be administered by the same or different routes, with respect to compositions described herein and the subject of the present disclosure.

Non-limiting examples of additional anti-inflammatory agents that may be employed include steroidal and non-steroidal compounds such as clobetasol propionate, betamethasone dipropionate, halobetasol proprionate, diflorasone diacetate, fluocinonide, halcinonide, amcinonide, desoximetasone, triamcinolone acetonide, mometasone furoate, fluticasone propionate, betamethasone dipropionate, fluocinolone acetonide, hydrocortisone valerate, hydrocortisone butyrate, flurandrenolide, triamcinolone acetonide, mometasone furoate, triamcinolone acetonide, fluticasone propionate, desonide, fluocinolone acetonide, hydrocortisone valerate, prednicarbate, triamcinolone acetonide, desonide, hydrocortisone, hydrocortisone aceponate, hydrocortisone buteprate, methylprednisolone aceponate, mometasone furoate and prednicarbate. Non-limiting examples of suitable non-steroidal anti-inflammatory compounds include indomethacin, ketoprofen, felbinac, diclofenac, ibuprofen, piroxicam, benzydamin, acetylsalicylic acid, diflunisal, salsalate, naproxen, fenoprofen, ketoprofen, flurbiprofen, oxaprozin, loxoprofen, indomethacin, sulindac, etodolac, ketorolac, diclo-fenac, nabumetone, piroxicam, meloxicam, tenoxicam, droxicam, lornoxicam, isoxicam, mefenamic acid, meclofenamic acid, flufenamic acid, tolfenamic acid, firocoxib, and licofelone, semi-synthetic glycosaminoglycosan ethers, flavanols, flavonoids, isoflavones and derivatives. The anti-inflammatory agent may be a suppressor of cytokine signalling such as, for example, cyclosporin A, 6-thioguanine, sulfasalazine, mesalamine (5-aminosalicylic acid), etanercept, prednisolone, or balsalazide.

The anti-infective agent may be any agent which treats an infection in a subject. In particular embodiments, the anti-infective agent is able to kill or inhibit the growth of an infectious organism which is capable of being transferred, in entirety or in part, between cells via an apoptotic body. Suitable anti-infective agents include, but are not limited to, an anti-viral agent, an anti-bacterial agent, an anti-protozoal agent, or a combination thereof.

Illustrative anti-viral agents include, but are not limited to, abacavir sulfate, acyclovir especially acyclovir sodium, adefovir, amantadine especially amantadine hydrochloride, amprenavir, ampligen, atazanavir, cidofovir, darunavir, delavirdine especially delavirdine mesylate, didanosine, docosanol, dolutegravir, edoxudine, efavirenz, emtricitabine, elvitegravir, enfuvirtide, entecavir, famciclovir, fomivirisen especially fomivirsen sodium, foscarnet especially foscarnet sodium, ganciclovir, ibacitabine, idoxuridine, imiquimod, indinavir especially indinavir sulfate, inosine pranobex, lamivudine, lopinavir, maraviroc, metisazone, moroxydine, nelfinavir especially nelfinavir mesylate, nevirapine, nitazoxanide, oseltamivir particularly oseltamivir phosphate, penciclovir, peramivir, pleconaril, podophyllotoxin, raltegravir, ribavirin, rimantadine especially rimantadine hydrochloride, ritonavir, saquinavir especially saquinavir mesylate, sofosbuvir, stavudine, telaprivir, tenofovir, tipranovir, trifluridine, tromantadine, umifenovir, valacyclovir especially valacyclovir hydrochloride, valganciclovir, vicriviroc, vidarabine, viramidine, zalcitabine, zanamivir, zidovudine and pharmaceutically acceptable salts and combinations thereof.

Illustrative anti-bacterial agents include, but are not limited to, quinolones (e.g. amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin, gemifloxacin, and garenoxacin), tetracyclines, glycylcyclines and oxazolidinones (e.g. chlortetracycline, demeclocycline, doxycycline, lymecycline, methacycline, minocycline, oxytetracycline, tetracycline, tigecycline; linezolide, eperezolid), glycopeptides, aminoglycosides (e.g. amikacin, arbekacin, butirosin, dibekacin, fortimicins, gentamicin, kanamycin, menomycin, netilmicin, ribostamycin, sisomicin, spectinomycin, streptomycin, tobramycin), β-lactams (e.g. imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephacetrile, cephalexin, cephaloglycin, cephaloridine, cephalothin, cephapirin, cephradine, cefinetazole, cefoxitin, cefotetan, azthreonam, carumonam, flomoxef, moxalactam, amdinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g. azithromycin, clarithromycin, erythromycin, oleandomycin, rokitamycin, rosaramicin, roxithromycin, troleandomycin), ketolides (e.g. telithromycin, cethromycin), coumermycins, lincosamides (e.g. clindamycin, lincomycin), chloramphenicol, clofazimine, cycloserine, dapsone, ethambutol hydrochloride, isoniazid, pyrazinamide, rifabutin, rifampin, rifapentine and streptomycin sulfate.

Illustrative anti-protozoal agents include, but are not limited to, atovaquone, metronidazole including metronidazole hydrochloride, pentamidine including pentamidine isethionate, chloroquine including chloroquine hydrochloride and chloroquine phosphate, doxycycline, hydroxychloroquine sulfate, mefloquine including mefloquine hydrochloride, primaquine including primaquine phosphate, pyrimethamine, pyrimethamine with sulfadoxine, trimethoprim, sulfamethoxazole, clindamycin, quinine, quinidine, sulfadiazine, artemether, lumefantrine, artesunate, nitazoxanide, suramin, melarsoprol, eflornithine, nifurtimox, stibogluconate including sodium stibogluconate, amphotericin B including liposomal amphotericin B, miltefosine, paromomycin, ketoconazole, itraconazole, fluconazole, and pharmaceutically acceptable salts and combinations thereof.

Illustrative immunosuppressive agents include, but are not limited to: corticosteroids such as, for example, budesonide, prednisone and prednisolone; mTOR inhibitors such as, for example, sirolimus and everolimus; and monoclonal antibodies such as, for example, certolizumab, ustekinumab and vedolizumab, and biosimilars thereof.

In exemplary embodiments the one or more Lactobacillus species described herein are provided and administered in the form of microbial biotherapeutic compositions. Such compositions may further comprise one or more additional microorganisms such as, for example, Lactobacillus rhamnosus, Lactobacillus plantarum, Lactobacillus bulgaricus, Lactobacillus casei, Lactobacillus acidophilus, Lactobacillus fermentum, Lactococcus lactis, Streptococcus thermophilus, Bifidobacterium breve, Bifidobacterium bifidum, Bifidobacterium lactis, Bifidobacterium animalis and Saccharomyces boulardii.

Microbial biotherapeutic compositions may comprise one or more prebiotic components. Suitable prebiotics include, for example, polydextrose, inulin, fructooligosaccharides (FOS), xylooligosaccharides (XOS), galactooligosaccharides (GOS), mannan oligosaccharides, protein-based green lipped mussel extract, and various prebiotic-containing foods such as raw onion, raw leek, raw chickory root and raw artichoke. In certain embodiments the prebiotic is a fructooligosaccharide.

Compositions comprising Lactobacillus species as described herein may be administered in any suitable form, including any of the dosage forms described above. The microbial biotherapeutic compositions may be provided to the user in a powder form, suitable for mixing by the user into any type of drink or food product (for example water, fruit juice or yoghurt) or for consumption as a powder in the absence of a drink or additional food product. The microbial biotherapeutic compositions may therefore be conveniently incorporated in a variety of food and/or beverage products, nutraceutical products, supplements, food additives, and over-the-counter formulations. The food or food additive may be a solid form such as a powder, or a liquid form. Specific examples of the types of beverages or foods include, but are not limited to water-based, milk-based, yoghurt-based, other dairy-based, milk-substitute based such as soy milk or oat milk, or juice-based beverages, water, soft drinks, carbonated drinks, and nutritional beverages, (including a concentrated stock solution of a beverage and a dry powder for preparation of such a beverage); baked products such as crackers, breads, muffins, rolls, bagels, biscuits, cereals, bars such as muesli bars, health food bars and the like, dressings, sauces, custards, yoghurts, puddings, pre-packaged frozen meals, soups and confectioneries.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1—Collagen Antibody-Induced Arthritis (CAIA) Mouse Model

In the present study the inventors used a validated mouse model of rheumatoid arthritis, the collagen antibody-induced arthritis (CAIA) model to compare the efficacy of a composition comprising three microbial biotherapeutic bacterial strains Lactobacillus paracasei (SVT 04P1), Lactobacillus buchneri (SVT 06B1) and Lactobacillus zeae (SVT 08Z1), with known treatments for rheumatoid arthritis (tofacitinib and adalimumab), and the effect of combining these known treatments with the microbial biotherapeutics.

Female 8-9 week old BALB/c mice were divided into seven groups:
Group 1—non-treatment (negative control) group (n=8).
Group 2—CAIA+vehicle (0.9% sterile saline+2.5% sucrose) (n=12).
Group 3—CAIA+combination of SVT 04P1, SVT 06B1 and SVT 08Z1, at a concentration of 3.0×10¹⁰ cfu/mL (n=12).
Group 4—CAIA+tofacitinib at a dose of 30 mg/kg (n=12).
Group 5—CAIA+tofacitinib at a dose of 30 mg/kg+combination of SVT 04P1, SVT 06B1 and SVT 08Z1 at a concentration of 3.0×10¹⁰ cfu/mL (n=12).
Group 6—CAIA+adalimumab a dose of 3 mg/kg (n=12).
Group 7—CAIA+adalimumab a dose of 3 mg/kg+combination of SVT 04P1, SVT 06B1 and SVT 08Z1 at a concentration of 3.0×10¹⁰ cfu/mL (n=12).

Arthritis was induced (CAIA) in groups 2 to 7 with a single 0.2 mL injection of Arthritomab (MD Biosciences) on day 0. Animals received a boost injection of LPS on day 6. Animals of Group 2 received vehicle (0.9% sterile saline+2.5% sucrose) by oral gavage daily from days 1 to 17 in a dose volume of 0.5 mL. Groups 3 to 7 received the test item daily from day 1 to day 17. The Lactobacillus combination (SVT 04P1, SVT 06B1 and SVT 08Z1) was administered (Groups 3, 5 and 7) by oral gavage in a dose volume of 0.5 mL. Tofacitinib was administered (Groups 4 and 5) by oral gavage in a dose volume of 10 mL/kg. Adalimumab was administered (Groups 6 and 7) was administered by subcutaneous injection in a dose volume of 10 mL/kg.

In-life observations were performed on all animals. Body weights were recorded once prior to dosing, and every other day after treatment initiation. Disease scoring was made once pretreatment, and the times a week starting on day 5. Paw volume of the hindlimbs was measured using a plethysmometer, and the sum of volumes was calculated. In brief, the plethysmometer is a volume meter, designed for accurate measurements of inflammation-induced swelling. It consists of a water filled cell into which the paw along with the ankle joint is dipped. A transducer records the differences in water level caused by volume displacement and provides LCD readout of the exact volume gain due to swelling.

Clinical score for the four limbs (score from 0 to 4) was evaluated based on the following scoring protocol: score 0—normal; score 1—erythema and mild swelling confined to the mid-foot (tarsals) or ankle joint or digits; score 2—erythema and mild swelling extending from the ankle to the mid-foot (2 segments); score 3—erythema and moderate swelling extending from the ankle to the metatarsal joints (2 segments); score 4—erythema and severe swelling encompassing the ankle, foot and digits.

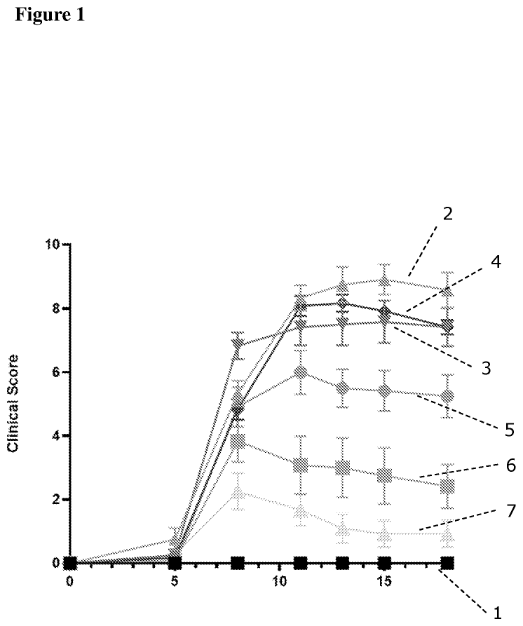

As shown in FIG. 1, clinical scores for mice treated with a combination of tofacitinib and the Lactobacillus strains (Group 5) were significantly lower from days 10 to 17 than mice treated with tofacitinib alone (Group 4). Clinical scores for mice treated with a combination of adalimumab and the Lactobacillus strains (Group 7) were significantly lower from days 8 to 17 than mice treated with adalimumab alone (Group 6).

### Deposit Details

Details of the biological material deposited pursuant to the Budapest Treaty are provided hereinbefore in the specification. The deposited strains have been previously described in international application no. PCT/AU2019/051092 (WO 2020/073088). In summary:

Lactobacillus parafarraginis SVT 05P2 was deposited pursuant to the Budapest Treaty with the Belgian Coordinated Collections of Microorganisms (BCCM), Federal Public Planning Service Science Policy, 8, rue de la Science B-1000, Brussels, Belgium, on 27 Feb. 2019 under Accession Number LMG P-31292.

Lactobacillus buchneri SVT 06B1 was deposited pursuant to the Budapest Treaty with the Belgian Coordinated Collections of Microorganisms (BCCM), Federal Public Planning Service Science Policy, 8, rue de la Science B-1000, Brussels, Belgium, on 27 Feb. 2019 under Accession Number LMG P-31293.

Lactobacillus zeae SVT 08Z1 was deposited pursuant to the Budapest Treaty with the Belgian Coordinated Collections of Microorganisms (BCCM), Federal Public Planning Service Science Policy, 8, rue de la Science B-1000, Brussels, Belgium, on 27 Feb. 2019 under Accession Number LMG P-31295.

L. rapi SVT 07R1 was deposited pursuant to the Budapest Treaty with the Belgian Coordinated Collections of Microorganisms (BCCM), Federal Public Planning Service Science Policy, 8, rue de la Science B-1000, Brussels, Belgium, on 27 Feb. 2019 under Accession Number LMG P-31294.

Lactobacillus paracasei SVT 04P1 was deposited pursuant to the Budapest Treaty with the Belgian Coordinated Collections of Microorganisms (BCCM), Federal Public Planning Service Science Policy, 8, rue de la Science B-1000, Brussels, Belgium, on 27 Feb. 2019 under Accession Number LMG P-31290.

Lactobacillus diolivorans SVT 01D1 was deposited pursuant to the Budapest Treaty with the Belgian Coordinated Collections of Microorganisms (BCCM), Federal Public Planning Service Science Policy, 8, rue de la Science B-1000, Brussels, Belgium, on 27 Feb. 2019 under Accession Number LMG P-31287.

1. A method for treating an inflammatory disorder of the joints or at least one symptom thereof, comprising administering to a subject in need thereof an effective amount of:
(i) an immunosuppressant; and
(ii) one or more Lactobacillus species selected from Lactobacillus buchneri, Lactobacillus paracasei, Lactobacillus zeae, Lactobacillus rapi, Lactobacillus parafarraginis, and Lactobacillus diolivorans, and/or a culture supernatant or cell free filtrate derived from culture media in which said one or more Lactobacillus species has been cultured.

2. A method according to claim 1, wherein the inflammatory disorder is an inflammatory arthritis.

3. A method according to claim 1, wherein the disorder is rheumatoid arthritis.

4. A method according to claim 1, wherein the immunosuppressant is a TNF inhibitor.

5. A method according to claim 4, wherein the TNF inhibitor is adalimumab.

6. A method according to claim 1, wherein the immunosuppressant is a JAK inhibitor.

7. A method according to claim 6, wherein the JAK inhibitor is tofacitinib.

8. A method according to claim 1, wherein the Lactobacillus species comprises a combination of Lactobacillus buchneri, Lactobacillus paracasei and Lactobacillus zeae.

9. A method according to claim 1, comprising administering to the subject an effective amount of adalimumab and a combination of Lactobacillus buchneri, Lactobacillus paracasei and Lactobacillus zeae.

10. A method according to claim 1, comprising administering to the subject an effective amount of tofacitinib and a combination of Lactobacillus buchneri, Lactobacillus paracasei and Lactobacillus zeae.

11. A method according to claim 1, wherein the immunosuppressant and the one or more Lactobacillus species, culture supernatant(s) or cell free filtrate(s) therefrom, are formulated in the same composition for administration.

12. A method according to claim 1, wherein the immunosuppressant and the one or more Lactobacillus species, culture supernatant(s) or cell free filtrate(s) therefrom, are formulated in different compositions for administration.

13. A method according to claim 12, wherein administration of the immunosuppressant and of the one or more Lactobacillus species, culture supernatant(s) or cell free filtrate(s) therefrom, is sequential or simultaneous.

14. A method of manufacturing a medicament for the treatment of an inflammatory disorder of the joints or at least one symptom thereof, the method comprising obtaining
(i) an immunosuppressant; and
(ii) one or more Lactobacillus species selected from Lactobacillus buchneri, Lactobacillus paracasei, Lactobacillus zeae, Lactobacillus rapi, Lactobacillus parafarraginis, and Lactobacillus diolivorans, and/or a culture supernatant or cell free filtrate derived from culture media in which said one or more Lactobacillus species has been cultured.
